(19) United States
(12) Patent Application Publication
(10) Pub. No.: US 2026/0083447 A1
(43) Pub. Date: Mar. 26, 2026

(54) MEDIAL COLLATERAL LIGAMENT RETRACTOR SYSTEMS AND METHODS

(71) Applicant: Optimotion Implants LLC, Orlando, FL (US)

(72) Inventors: Vuong Binh NGUYEN, Windermere, FL (US); Michael MESSIEH, Clermont, FL (US); Cassidy LARWOOD, Oviedo, FL (US); Andrew RYNEARSON, Winter Springs, FL (US)

(73) Assignee: Optimotion Implants LLC, Orlando, FL (US)

(21) Appl. No.: 19/183,300

(22) Filed: Apr. 18, 2025

Related U.S. Application Data
(60) Provisional application No. 63/636,000, filed on Apr. 18, 2024.

(51) Int. Cl. A61B 17/02; A61B 17/15; A61B 17/17; A61B 34/10; A61B 34/20

(52) U.S. Cl. CPC A61B 17/025; A61B 17/155; A61B 17/1764; A61B 34/10; A61B 34/20; A61B 2017/0268; A61B 2034/108; A61B 2034/2051; A61B 2034/2055

(57) ABSTRACT

A ligament retractor system may include a ligament retractor, one or more sensors configured to identify a location of one or more anatomical landmarks or instruments, and a processing unit configured to receive and process data from the one or more sensors.

### CROSS REFERENCE TO RELATED APPLICATIONS

The present application claims the benefit of U.S. Provisional Patent Application Ser. No. 63/636,000, filed on Apr. 18, 2024 and entitled “MEDIAL COLLATERAL LIGAMENT RETRACTOR SYSTEMS AND METHODS”, which is incorporated by reference as though set forth herein in its entirety.

### TECHNICAL FIELD

The present disclosure relates to systems and methods for use in orthopedic surgery. More specifically, the present disclosure relates to ligament retractor systems and methods.

### BACKGROUND

Total knee arthroplasty (TKA) is a widely performed surgical procedure aimed at restoring function and alleviating pain in patients suffering from advanced knee arthritis or joint degeneration. The success of a TKA procedure heavily relies on precise bone resections and soft tissue management to achieve optimal alignment, balance, and fit of a prosthetic component. Conventional methods for performing bone resections involve the use of cutting guides and surgeon experience to achieve the desired resection angles and depths. However, even with computer-assisted navigation and robotic-assisted surgery, there remains a need for improved accuracy and real-time intraoperative verification of bone cuts.

Patient-specific instrumentation (PSI) has emerged as a method to enhance surgical accuracy by creating customized cutting blocks based on preoperative imaging. These blocks are designed to conform to a patient's unique anatomy, reducing reliance on traditional alignment guides. However, existing PSI solutions lack real-time intraoperative feedback to ensure that bone cuts are performed precisely as planned.

To overcome these challenges, there is a need for real-time tracking and visualization tools, that can provide surgeons with immediate feedback on bone cuts and implant positioning.

### SUMMARY

The various systems and methods of the present disclosure have been developed in response to the present state of the art, and in particular, in response to the problems and needs in the art that have not yet been fully solved by currently available ligament retractor systems and methods.

In some embodiments, a ligament retractor system may include a ligament retractor, one or more sensors configured to identify a location of one or more anatomical landmarks or instruments, and a processing unit configured to receive and process data from the one or more sensors.

In the ligament retractor system of any preceding paragraph, the ligament retractor system may further include a patient specific instrument (PSI) retractor attachment configured to be received by, and operatively engage, the ligament retractor, wherein the PSI retractor may include the one or more sensors.

In the ligament retractor system of any preceding paragraph, the ligament retractor may include a medial collateral ligament (MCL) retractor and the one or more anatomical landmarks may include at least one of a tibial plateau and a soft tissue adjacent to the tibial plateau.

In the ligament retractor system of any preceding paragraph, the one or more sensors may include an inertial measurement unit (IMU) sensor configured to obtain positional data of the ligament retractor.

In the ligament retractor system of any preceding paragraph, the one or more sensors may include an optical sensor configured to provide 3D visualization data of an anatomical landmark proximate the ligament retractor.

In the ligament retractor system of any preceding paragraph, the one or more sensors may include a Hall effect sensor configured to detect proximity of a metallic surgical instrument to the ligament retractor.

In the ligament retractor system of any preceding paragraph, the ligament retractor may include the one or more sensors.

In some embodiments, a ligament retractor system may include a ligament retractor that may include one or more sensors, and a processing unit configured to receive and process data from the one or more sensors. The one or more sensors may be configured to detect proximity of a cutting device to the one or more sensors.

In the ligament retractor system of any preceding paragraph, the one or more sensors may include one or more Hall effect sensors.

In the ligament retractor system of any preceding paragraph, the cutting device may include a material having a magnetic field detectable by the one or more sensors.

In the ligament retractor system of any preceding paragraph, the ligament retractor system may further include a cutting guide. The cutting guide may be configured to be coupled to the ligament retractor and to receive and guide the cutting device.

In the ligament retractor system of any preceding paragraph, the ligament retractor system may further include a patient specific instrument (PSI) retractor attachment configured to be received by, and operatively engage, the ligament retractor.

In the ligament retractor system of any preceding paragraph, the ligament retractor system may further include a cutting guide, wherein the cutting guide may be configured to be coupled to the PSI retractor attachment and to receive and guide the cutting device.

In the ligament retractor system of any preceding paragraph, the ligament retractor may include a medial collateral ligament (MCL) retractor.

In the ligament retractor system of any preceding paragraph, the ligament retractor system may further include a bone saw having a saw blade and a battery pack configured to provide electric voltage to power the bone saw. The battery pack may include an indicator and a circuit board configured to receive a trigger voltage from the one or more sensors, the one or more sensors may be configured to communicate the trigger voltage to the circuit board upon detection of the proximity of the saw blade, the circuit board may be configured to activate the indicator upon receipt of the trigger voltage, and the circuit board may be further configured to switch off the electric voltage from the battery pack to the bone saw upon receipt of the trigger voltage.

In some embodiments, a ligament retractor system may include a ligament retractor that may include one or more sensors, and a processing unit configured to receive and process data from the one or more sensors. The one or more sensors may be configured to confirm placement of the ligament retractor during a surgical procedure.

In the ligament retractor system of any preceding paragraph, the one or more sensors may include one or more inertial measurement unit (IMU) sensors configured to obtain positional data of the ligament retractor.

In the ligament retractor system of any preceding paragraph, the ligament retractor may include a medial collateral ligament (MCL) retractor.

In the ligament retractor system of any preceding paragraph, the ligament retractor may further include a Hall effect sensor and the ligament retractor system may further include a cutting device, wherein the Hall effect sensor may be configured to detect a proximity of the cutting device to the ligament retractor.

In the ligament retractor system of any preceding paragraph, the cutting device may include a material having a magnetic field detectable by the one or more sensors.

In some embodiments, a ligament retractor system may include a ligament retractor configured to retract a ligament during a surgical procedure, and a patient specific instrument (PSI) retractor attachment configured to be received by, and operatively engage, the ligament retractor. The PSI retractor attachment may be configured to engage an anatomical landmark.

In the ligament retractor system of any preceding paragraph, the ligament retractor may include one or more sensors configured to identify a location of one or more anatomical landmarks or instruments.

In the ligament retractor system of any preceding paragraph, the PSI retractor attachment may include one or more sensors configured to identify a location of one or more anatomical landmarks or instruments.

In the ligament retractor system of any preceding paragraph, the ligament retractor system may further include a cutting guide. The cutting guide may be configured to be coupled to the PSI retractor attachment and to receive and guide a cutting device.

These and other features and advantages of the present disclosure will become more fully apparent from the following description and appended claims or may be learned by the practice of the implants, systems, and methods set forth hereinafter.

### BRIEF DESCRIPTION OF THE DRAWINGS

Exemplary embodiments of the disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the disclosure will be described with additional specificity and detail through use of the accompanying drawings.

FIG. 1 is a perspective view of a medial collateral ligament (MCL) retractor system, an exemplary portion of a tibia, and an exemplary saw blade according to an embodiment of the present disclosure.

FIG. 2 is a perspective view of an MCL retractor, a patient specific instrument (PSI) retractor attachment, and an exemplary portion of a tibia according to an embodiment of the present disclosure.

FIG. 3 is a perspective view of an MCL retractor, a PSI retractor attachment, and an exemplary portion of a tibia according to an embodiment of the present disclosure.

FIG. 4 is a perspective view of an MCL retractor including a retractor fiducial array, an exemplary portion of a tibia, and an exemplary portion of a femur according to an embodiment of the present disclosure.

FIG. 5 is a perspective view of an MCL retractor including a retractor fiducial array, a PSI retractor attachment, an exemplary portion of a tibia, and an exemplary portion of a femur according to an embodiment of the present disclosure.

FIG. 6 is a perspective view of an MCL retractor, a PSI retractor attachment, a cutting guide, and an exemplary portion of a tibia according to an embodiment of the present disclosure.

FIG. 7 is a perspective view of an MCL retractor including a retractor fiducial array, a PSI retractor attachment, a cutting guide including a cutting guide fiducial array, and an exemplary portion of a tibia according to an embodiment of the present disclosure.

FIG. 8 is a perspective view of an MCL retractor, a PSI retractor attachment, and an exemplary portion of a tibia according to an embodiment of the present disclosure.

FIG. 9 is a perspective view of an MCL retractor, a PSI retractor attachment, a cutting guide, and an exemplary portion of a tibia according to an embodiment of the present disclosure.

FIG. 10 is a perspective view of an MCL retractor, a PSI retractor attachment, a cutting guide, an exemplary saw blade, and an exemplary portion of a tibia according to an embodiment of the present disclosure.

FIG. 11 is a perspective view of an MCL retractor including sensors and Hall sensors, and an exemplary saw blade.

FIG. 12 is a perspective view of a representative sensor, a representative PSI retractor attachment, and an exemplary portion of a tibia according to an embodiment of the present disclosure.

FIG. 13A is a perspective view of an MCL retractor housing according to an embodiment of the present disclosure.

FIG. 13B is a perspective view of an MCL retractor insert according to an embodiment of the present disclosure.

FIG. 14 is a perspective view of a cutting guide engaged with an exemplary portion of a femur according to an embodiment of the present disclosure.

FIG. 15A is a perspective view of a circuit board and an indicator according to an embodiment of the present disclosure.

FIG. 15B is a perspective view of a battery pack according to an embodiment of the present disclosure.

FIG. 15C is a perspective view of an exemplary bone saw according to an embodiment of the present disclosure.

FIG. 16 shows three perspective views of an exemplary portion of a tibia and an exemplary portion of a femur indicating anatomical landmarks and a homerun wire according to an embodiment of the present disclosure.

FIG. 17 is an annotated radiographic image of an exemplary portion of a tibia indicating anatomical landmarks according to an embodiment of the present disclosure.

FIG. 18 is a graphical representation of a portion of a tibia indicating potential resection planes in relation to placement of a homerun wire according to an embodiment of the present disclosure.

FIG. 19 is a graphical representation of a portion of a tibia indicating exemplary angles between various anatomical landmarks.

FIG. 20 is an annotated radiographic image of an exemplary portion of a tibia indicating anatomical landmarks according to an embodiment of the present disclosure.

FIG. 21 is an annotated radiographic image of an exemplary lateral femoral condyle and an exemplary medial femoral condyle.

FIG. 22 is a superior view of an exemplary distal femur indicating anatomical landmarks.

FIG. 23 is a superior view of an exemplary distal femur indicating anatomical landmarks.

FIG. 24 is a section of a superior view of an exemplary distal femur indicating anatomical landmarks and a Nguyen Messieh Line according to an embodiment of the present disclosure.

FIG. 25 is an annotated radiographic image of an exemplary distal femur indicating anatomical landmarks and a Nguyen Messieh Line according to an embodiment of the present disclosure.

FIG. 26 is an annotated radiographic image of an exemplary distal femur indicating anatomical landmarks and a Nguyen Messieh Line according to an embodiment of the present disclosure.

FIG. 27 is an annotated radiographic image of an exemplary distal femur indicating anatomical landmarks.

FIG. 28 is an annotated radiographic image of an exemplary distal femur indicating anatomical landmarks.

FIG. 29 is an example of a kinematically aligned robotic plan.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

The present disclosure relates to medial collateral ligament (MCL) retractor devices, systems, and methods. Those skilled in the art will recognize that the following description is merely illustrative of the principles of the technology, which may be applied in various ways to provide many alternative embodiments. The present disclosure illustrates devices for an MCL retractor for the purposes of illustrating the concepts of the present design. However, it will be understood that other variations and uses are contemplated including, but not limited to, a ligament retractor system including: collateral ligament retractors, concave total knee retractors, posterior condylar retractors, PCL retractors, proximal tibial retractors, other retractors used in total knee arthroplasty surgical procedures, retractors used in other total joint arthroscopy surgical procedures, other retractors configured to retract a ligament during a surgical procedure, etc.

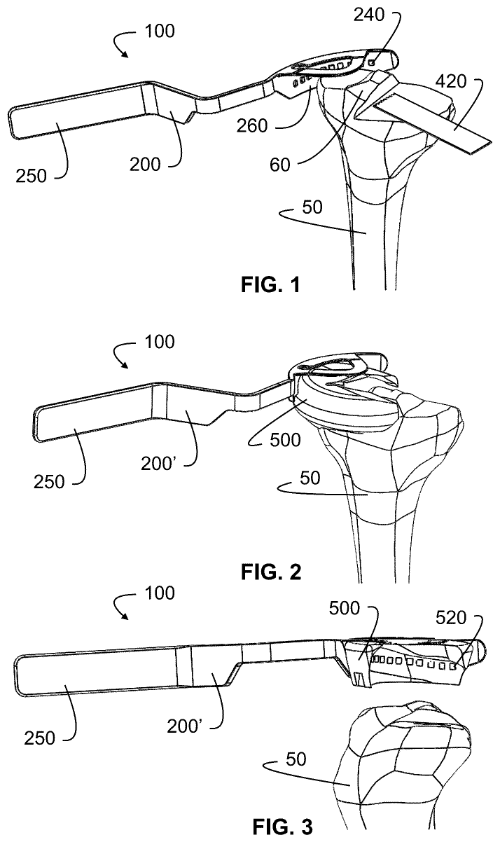

FIG. 1 is a perspective view of a medial collateral ligament (MCL) retractor system 100, an exemplary portion of a tibia 50, and an exemplary saw blade 420 according to an embodiment of the present disclosure. The MCL retractor system 100 may include a medial collateral ligament (MCL) retractor 200 configured to protect the medial ligament and/or lateral ligament during knee arthroplasty surgery. The MCL retractor 200 may be configured to spread a lateral ligament and/or a medial ligament during tibial resection. Additionally, or alternatively, the MCL retractor 200 may be configured to help in positioning a prosthetic implant.

The MCL retractor 200 may include a handle portion 250 and a bone engaging portion 260. The bone engaging portion 260 may include one or more Hall effect sensors 240. The one or more Hall effect sensors 240 may be electrically connected to an external device such as a bone saw 400. Additionally, or alternatively, the one or more Hall effect sensors 240 may be connected to an external device configured to alert a user if a cutting device is within a predetermined proximity to one or more Hall effect sensors 240. The cutting device may include a material having a magnetic field detectable be one or more sensors. The one or more Hall effect sensors 240 may be employed to protect the medial collateral ligament (MCL) when utilizing a lateral approach for knee arthroplasty surgery.

The external device may be configured to alert a user via a light indicator, a sound indicator, a haptic indicator, or a combination of one or more of the above. The predetermined proximity may be 10 mm, 8 mm, 6 mm, 4 mm, 2 mm, 1 mm or any value within a range of 1 mm to 10 mm.

The one or more Hall effect sensors 240 may be configured as an array of proximity sensors configured to detect a position of a cutting device such as a saw blade 420, drill, burr, reamer, and/or other bone cutting device. The one or more Hall effect sensors 240 may be positioned to have a functional sensing field that may encompass a tibial plateau 60.

The one or more Hall effect sensors 240 may be contactless proximity sensors switches configured to detect a magnetic field and provide either digital or analog outputs. The one or more Hall effect sensors 240 may be either linear unipolar or bipolar and may respond to the polarity of a magnetic field. The one or more Hall effect sensors 240 may be configured to detect ferrous metals. The output from the one or more Hall effect sensors 240 may be connected to an amplifier and/or potentiometer to measure the threshold signal. The threshold signal may be used for light, audio alerts, or as an off/on power switch.

The one or more Hall effect sensors 240 may be located on a target area near the MCL. The one or more Hall effect sensors 240 may be positioned parallel to a saw blade during a surgical procedure. This positioning may allow for real-time monitoring and detection of any magnetic or ferrous materials in a tissue protector to avoid a threat to the MCL. The output of the one or more Hall effect sensors 240 may trigger alerts or safety measures to prevent potential damage.

FIG. 2 is a perspective view of an MCL retractor 200′, a patient specific instrument (PSI) retractor attachment 500, and an exemplary portion of a tibia 50 according to an embodiment of the present disclosure. A bone engaging portion 260 of an MCL retractor 200′ may be configured to receive a PSI retractor attachment 500. The PSI retractor attachment 500 may be patient specific and may be designed based on CT, MRI, and/or other radiographic images of a patient. The PSI retractor attachment 500 may be fabricated using 3D printing and/or other additive manufacturing processes. The PSI retractor attachment 500 may be configured to engage an articulating surface of a medial tibial plateau, an articulating surface of a lateral tibial plateau, and/or an anatomical landmark.

FIG. 3 is a perspective view of an MCL retractor 200′, a PSI retractor attachment 500, and an exemplary portion of a tibia 50 according to an embodiment of the present disclosure. The PSI retractor attachment 500 may include one or more Hall effect sensors 520. The one or more Hall effect sensors 520 may be electrically connected to an external device such as a bone saw 400. Additionally, or alternatively, the one or more Hall effect sensors 520 may be connected to an external device configured to alert a user if a cutting device is within a predetermined proximity to one or more Hall effect sensors 520.

The external device may be configured to alert a user via a light indicator, a sound indicator, a haptic indicator, or a combination of one or more of the above. The predetermined proximity may be 10 mm, 8 mm, 6 mm, 4 mm, 2 mm, 1 mm or any value within a range of 1 mm to 10 mm.

The one or more Hall effect sensors 520 may be configured as an array of proximity sensors configured to detect a position of a cutting device such as a saw blade 420, drill, burr, reamer, and/or other bone cutting device. The one or more Hall effect sensors 520 may be positioned to have a functional sensing field that may encompass a tibial plateau 60.

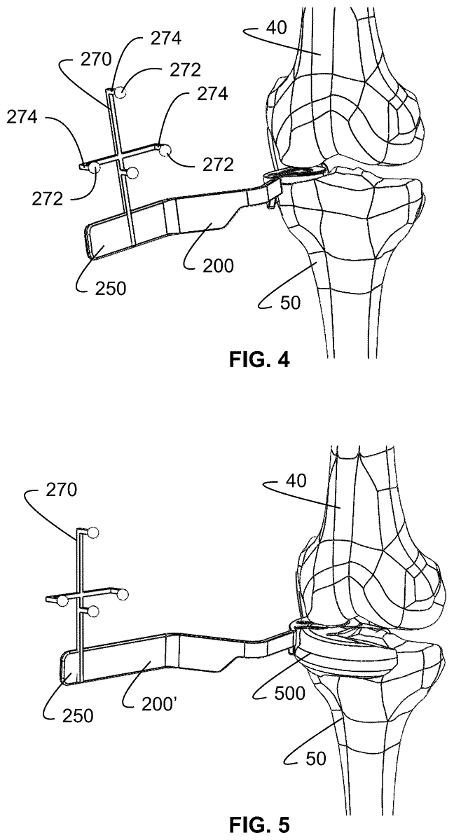

FIG. 4 is a perspective view of an MCL retractor 200 including a retractor fiducial array 270, an exemplary portion of a tibia 50, and an exemplary portion of a femur 40 according to an embodiment of the present disclosure. The handle portion 250 of the MCL retractor 200 may include a retractor fiducial array 270. The retractor fiducial array 270 may be configured to facilitate real-time trajectory alignment, relative position, and/or relative motion tracking of the MCL retractor system 100 by a surgical navigation system and/or a robotic assisted surgical system.

The retractor fiducial array 270 may include a plurality of arms 274 and a plurality of locators 272. The plurality of locators 272 may be positioned at the ends of the plurality of arms 274. The plurality of locators 272 may be configured as reference features to facilitate real-time trajectory alignment, relative position, and/or relative motion tracking of the MCL retractor system 100 by a surgical navigation system and/or a robotic assisted surgical system.

The retractor fiducial array 270 may be configured to facilitate and/or confirm correct placement of the MCL retractor 200 such that the MCL retractor 200 may be placed deep to the MCL and may protect the MCL from damage during a surgical procedure. The placement of the MCL retractor 200 may be facilitated and/or confirmed by a surgical navigation system and/or a robotic assisted surgical system.

FIG. 5 is a perspective view of an MCL retractor 200′ including a retractor fiducial array 270, a PSI retractor attachment 500, an exemplary portion of a tibia 50, and an exemplary portion of a femur 40 according to an embodiment of the present disclosure. The handle portion 250 of the MCL retractor 200′ may include a retractor fiducial array 270. The retractor fiducial array 270 may be configured to facilitate real-time trajectory alignment, relative position, and/or relative motion tracking of the MCL retractor system 100 by a surgical navigation system and/or a robotic assisted surgical system.

The retractor fiducial array 270 may be configured to facilitate and/or confirm correct placement of the MCL retractor 200′ such that the MCL retractor 200′ may be placed deep to the MCL and may protect the MCL from damage during a surgical procedure. The placement of the MCL retractor 200′ may be facilitated and/or confirmed by a surgical navigation system and/or a robotic assisted surgical system.

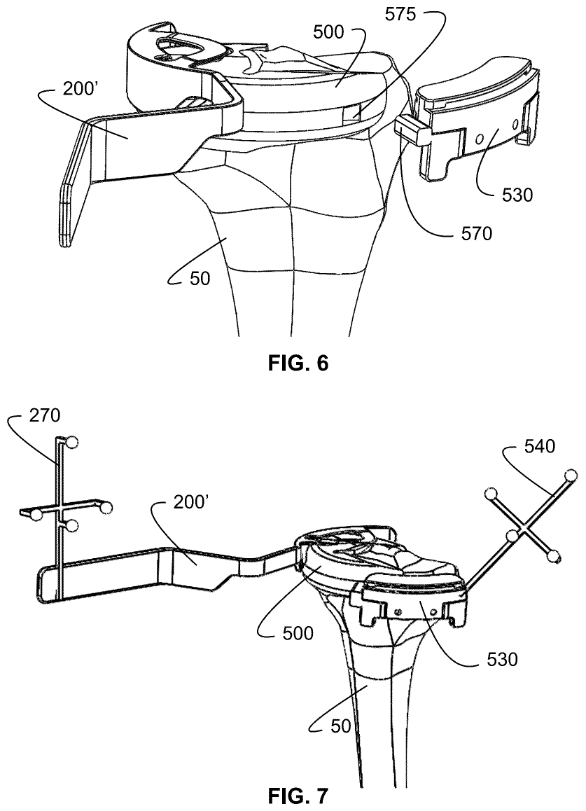

FIG. 6 is a perspective view of an MCL retractor 200′, a PSI retractor attachment 500, a cutting guide 530, and an exemplary portion of a tibia 50 according to an embodiment of the present disclosure. The MCL retractor system 100 may include a cutting guide 530 configured to guide a cutting device to precisely and accurately resect a portion of bone. The cutting guide 530 may be patient specific and may be designed based on CT, MRI, and/or other radiographic images of a patient. The cutting guide 530 may be fabricated using 3D printing and/or other additive manufacturing processes. The cutting guide 530 may be configured to be coupled to the MCL retractor 200′ and to receive and guide the cutting device. Additionally, or alternatively, the cutting guide 530 may be configured to be coupled to the PSI retractor attachment 500 and to receive and guide the cutting device.

The cutting guide 530 may include a locating boss 570. The PSI retractor attachment 500 may include a locating aperture 575. The locating boss 570 and the locating aperture 575 may be configured to position the cutting guide 530 relative to the PSI retractor attachment 500. The locating boss 570 and the locating aperture 575 may further be configured to secure the cutting guide 530 to the PSI retractor attachment 500.

FIG. 7 is a perspective view of an MCL retractor 200′ including a retractor fiducial array 270, a PSI retractor attachment 500, a cutting guide 530 including a cutting guide fiducial array 540, and an exemplary portion of a tibia 50 according to an embodiment of the present disclosure. The cutting guide 530 may include a cutting guide fiducial array 540. The cutting guide fiducial array 540 may be configured to facilitate and/or confirm correct placement of the cutting guide 530. The placement of the cutting guide 530 may be facilitated and/or confirmed by a surgical navigation system and/or a robotic assisted surgical system.

Additionally, or alternatively, the retractor fiducial array 270 may be configured with one or more optical sensors and/or one or more inertial sensors. Additionally, or alternatively, the cutting guide fiducial array 540 may be configured with one or more optical sensors and/or one or more inertial sensors. The one or more optical sensors and/or one or more inertial sensors may be compatible with a surgical navigation system and/or a robotic assisted surgical system.

A method for resecting a portion of bone during a knee arthroplasty surgical procedure may include using an MCL retractor 200′, a PSI retractor attachment 500 and a cutting guide 530. A method for resecting a portion of bone during a knee arthroplasty surgical procedure may include the following steps:

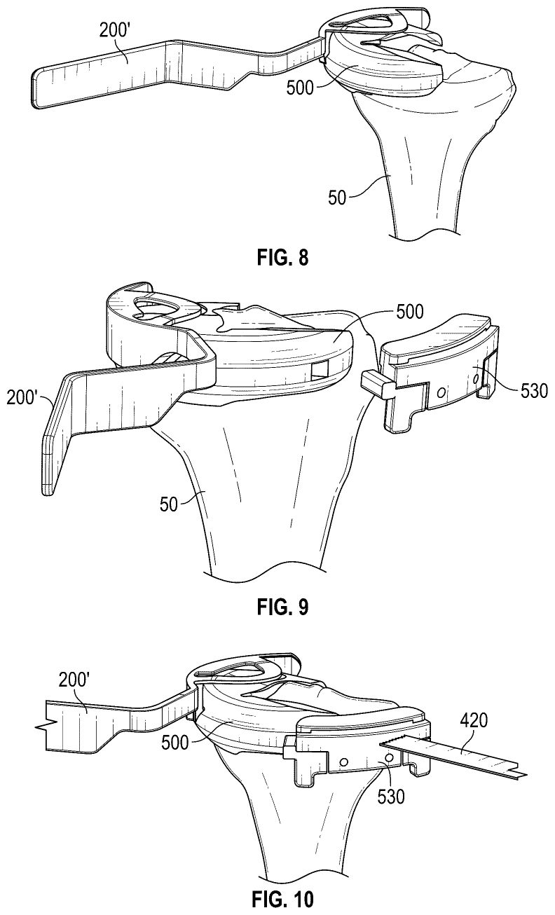

FIG. 8 is a perspective view of an MCL retractor 200′, a PSI retractor attachment 500, and an exemplary portion of a tibia 50 according to an embodiment of the present disclosure.

Step 1 may include attaching a PSI retractor attachment 500 to an MCL retractor 200′

FIG. 9 is a perspective view of an MCL retractor 200′, a PSI retractor attachment 500, a cutting guide 530, and an exemplary portion of a tibia 50 according to an embodiment of the present disclosure.

Step 2 may include placing the MCL retractor 200′ in the correct position to shield the MCL using the PSI retractor attachment 500 to confirm the placement. Correct placement may be confirmed when the PSI retractor attachment 500 is an exact negative fit to a patient's tibial anatomy.

FIG. 10 is a perspective view of an MCL retractor 200′, a PSI retractor attachment 500, a cutting guide 530, an exemplary saw blade 420, and an exemplary portion of a tibia 50 according to an embodiment of the present disclosure.

Step 3 may include attaching the cutting guide 530 to the PSI retractor attachment 500 and securing the placement of the cutting guide 530 with one or more pins.

Step 4 may include performing a tibial cut.

Placement of the MCL retractor 200′ and PSI retractor attachment 500 may be confirmed with optical and/or inertial sensors as described above.

As described above, the one or more Hall effect sensors 520 may detect the proximity of a saw blade 420 and/or other bone cutting device while cuts are being performed.

Those of skill in the art will recognize that this is only one of many potential methods that may be used to resect a portion of bone during a knee arthroplasty surgical procedure. In alternative embodiments, different devices may be used to resect a portion of bone during a knee arthroplasty surgical procedure using the methods described above. Further, the method set forth in FIG. 8 though FIG. 10 may be used in conjunction with other MCL retractor systems besides those specifically disclosed herein.

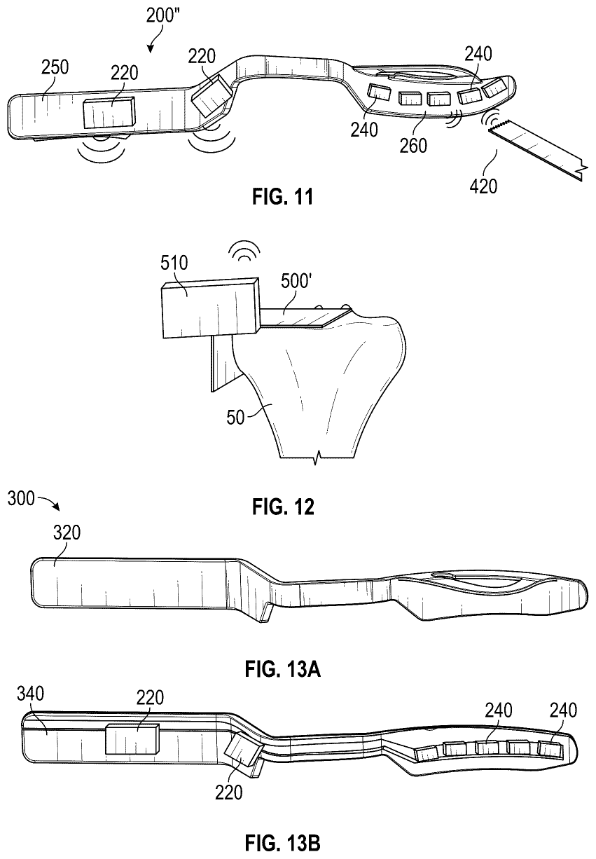

FIG. 11 is a perspective view of an MCL retractor 200″ including one or more sensors 220 and one or more Hall effect sensors 240, and an exemplary saw blade 420. The one or more sensors may be configured as optical sensors, inertial sensors, and/or a combination of optical and inertial sensors. The one or more sensors 220 may be located along a handle portion 250 of the MCL retractor 200″. The one or more sensors 220 may be configured to identify a location of one or more anatomical landmarks and/or instruments. The one or more anatomical landmarks may include a tibial plateau and/or a soft tissue adjacent to the tibial plateau. The one or more Hall effect sensors 240 may be located along a bone engaging portion 260 of the MCL retractor 200″.

At least one of the one or more sensors 220 may include an inertial measurement unit (IMU) sensor. The IMU sensor may be configured to obtain data related to the orientation, acceleration, and/or positional data of the MCL retractor 200″.

At least one of the one or more sensors 220 may include an optical sensor. The optical sensor may be configured to provide 3D visualization data of at least a portion of the tibial plateau 60 including soft tissues, and/or another anatomical landmark proximate the MCL retractor 200″. The provided data may facilitate targeted surgical interventions.

At least one of the one or more sensors 220 may include a Hall effect sensor 240. The Hall effect sensor 240 may be configured to detect proximity of a metallic surgical instrument to the MCL retractor 200″ and/or in relation to vital anatomical landmarks, and may enhance intraoperative safety measures.

The MCL retractor system 100 may include a processing unit configured to receive and process data received from the one or more sensors 220 and/or the hall effect sensors 240. Additionally, the processing unit may include a screen or other means for outputting processed data. Additionally, or alternatively, the processing unit may be configured to connect to an external display device and may generate information, images, warning, and/or other processed data to be presented by the external display device.

The collaborative function of an IMU sensor, an optical sensor, and/or a Hall effect sensor may provide a holistic surgical navigation system that may exceed the limitations inherent within single-sensor technologies. The combination of an IMU sensor, an optical sensor, and/or a Hall effect sensor may address the intricate challenges associated with knee arthroplasty, promising meticulous tissue management and minimizing the risk of unintended structural compromise.

The emphasis on an integrated processing unit may illuminate the capacity of the MCL retractor system 100 to support informed, real-time surgical decisions through comprehensive data analysis. The integration of an IMU sensor, an optical, and/or a Hall effect sensor within a unified system may harnessing the unique benefits of each sensor type to address the complex demands of surgery.

The one or more sensors 220 may be configured to increase safety and decrease the chances of damaging an MCL during a knee arthroplasty utilizing a lateral approach. The one or more sensors 220 may be configured to confirm correct orientation and placement of the an MCL retractor 200″. The one or more sensors 220 may further be configured to map a portion of a tibia 50 with ligament representation, when used in combination with pre-op MRI and/or CT scan data.

The one or more Hall effect sensors 240 may be configured to detect proximity of a saw blade 420, or other bone cutting device, to the MCL retractor 200″. The one or more Hall effect sensors 240 may be configured to communicate with a battery pack 440 of a bone saw 400.

FIG. 12 is a perspective view of a representative sensor 510, a representative PSI retractor attachment 500′, and an exemplary portion of a tibia 50 according to an embodiment of the present disclosure. One or more sensors 510 may be configured as optical sensors, inertial sensors, and/or a combination of optical and inertial sensors. The one or more sensors 510 may be fixed on a portion of a tibia 50 and may orient an MCL retractor 200″. Additionally, or alternatively, the one or more sensors 510 may be configured to map a portion of a tibia 50 with ligaments. The mapped portion of the tibia may be based on a patient 3D model, which may be generated from MRI and/or CT scan data, and intraoperative tibial registration points. The one or more sensors 510 may be fixed on a portion of the tibia via the PSI retractor attachment 500′.

The one or more sensors 510 may be configured to communicate with one or more sensors 220 fixed to the MCL retractor 200″ to confirm the location and orientation of the MCL retractor 200″ with respect to the anatomical features of the tibia as mapped by the one or more sensors 510.

FIG. 13A is a perspective view of an MCL retractor housing 320 according to an embodiment of the present disclosure. FIG. 13B is a perspective view of an MCL retractor insert 340 according to an embodiment of the present disclosure. An MCL retractor assembly 300 may include the MCL retractor housing and the MCL retractor insert 340. The MCL retractor insert 340 may include one or more sensors 220 and one or more Hall effect sensors 240. The MCL retractor insert 340 may be patient specific and may be designed based on CT, MRI, and/or other radiographic images of a patient. The MCL retractor insert 340 may be fabricated using 3D printing and/or other additive manufacturing processes.

The MCL retractor housing 320 may be configured to receive the MCL retractor insert 340. The MCL retractor insert 340 may be secured within the MCL retractor housing 320 through a snap fit, a plurality of clips, fasteners, or other means known in the art. The MCL retractor housing may be re-useable and may be fabricated using biocompatible materials suitable for autoclave sterilization, for example: stainless steel, PEEK, Radel, Delrin, or other material known in the art. The MCL retractor insert 340, the one or more sensors 220, and the one or more Hall effect sensors 240 may be disposable. Once assembled, the MCL retractor assembly 300 may have features as described above for the MCL retractor 200″.

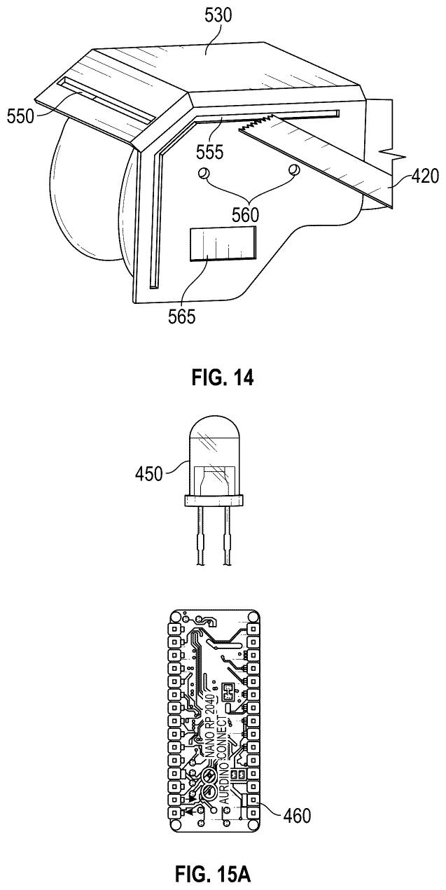

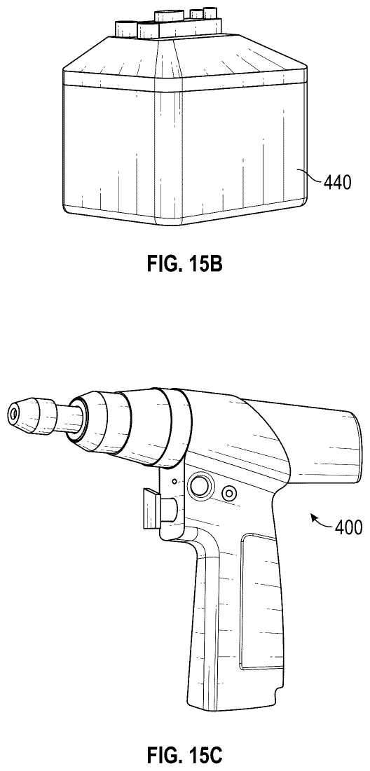

FIG. 14 is a perspective view of a cutting guide 530 engaged with an exemplary portion of a femur 40 according to an embodiment of the present disclosure. The cutting guide 530 may include a distal cut slot 550, one or more lateral cut slots 555, one or more pin holes 560, and a sensor pocket 565. The cutting guide 530 may be configured to guide a bone cutting device to precisely and accurately resect a portion of bone. The cutting guide 530 may be patient specific and may be designed based on CT, MRI, and/or other radiographic images of a patient. The cutting guide 530 may be fabricated using 3D printing and/or other additive manufacturing processes.

The distal cut slot 550 may be configured to receive a saw blade 420 and may be aligned with a distal resection plane of a portion of a tibia 50 and/or a femur 40. The one or more lateral cut slots 555 may be configured to receive a saw blade 420 and may be aligned with a lateral resection plane of a portion of a tibia 50 and/or a femur 40. The distal resection plane and the lateral resection plane may be identified during per-op planning and may be configured to modify a portion of a tibia and/or a femur 40 in preparation for receiving a prosthetic implant.

Alternatively, the distal cut slot 550 may be configured to receive a drill, burr, reamer, or other rotating cutting device and may be aligned with a distal resection plane of a portion of a tibia 50 and/or a femur 40. The one or more lateral cut slots 555 may be configured to receive a drill, burr, reamer, or other rotating cutting device and may be aligned with a lateral resection plane of a portion of a tibia 50 and/or femur 40.

The one or more pin holes 560 may be configured to allow the cutting guide 530 to be secured to a portion of the tibia 50 and/or a femur 40 and may prevent movement of the cutting guide 530 during resection of a portion of the tibia 50 and/or femur 40. The one or more pin holes 560 may be configured to receive a k-wire and/or a guide wire. One or more or the pin holes 560 may be located and configured to receive a homerun wire 600.

The sensor pocket 565 may be configured to receive one or more sensors. The one or more sensors may be configured to facilitate and/or confirm correct placement of the cutting guide 530. The placement of the cutting guide 530 may be facilitated and/or confirmed by a surgical navigation system and/or a robotic assisted surgical system.

FIG. 15A is a perspective view of a circuit board 460 and an indicator 450 according to an embodiment of the present disclosure. FIG. 15B is a perspective view of a battery pack 440 according to an embodiment of the present disclosure. FIG. 15C is a perspective view of an exemplary bone saw 400 according to an embodiment of the present disclosure.

The battery pack 440 may include the circuit board 460 and the indicator 450. The battery pack 440 may be rechargeable and may be configured to provide electrical power to activate the bone saw 400. The circuit board 460 may be electrically connected to the indicator 450. The circuit board may further be electrically connected to one or more Hall effect sensors 240 and/or one or more Hall effect sensors 520.

The circuit board 460 may be configured to receive a trigger voltage signal from the one or more Hall effect sensors 240 and/or one or more Hall effect sensors 520 if a cutting device is within a predetermined proximity to the one or more Hall effect sensors 240 and/or one or more Hall effect sensors 520. The circuit board 460 may be further configured to activate the indicator 450 upon receipt of a trigger voltage signal from the one or more Hall effect sensors 240 and/or one or more Hall effect sensors 520. The indicator 450 may be configured to alert a user via a light indicator, a sound indicator, a haptic indicator, or a combination of one or more of the above.

Additionally, or alternatively, the circuit board 460 may be configured to switch off the electric voltage to the bone saw 400 upon receipt of a trigger voltage signal from the one or more Hall effect sensors 240 and/or one or more Hall effect sensors 520. The trigger voltage level may be predetermined with calibration to relate sensor voltage output to saw blade distance. The desired saw blade distance may be set and programmed.

A Modular Patient-Specific Instrument (PSI) system 700 may include with at least one homerun wire 600. The modular PSI system 700 may allow for accurate placement of at least one homerun wire 600 in accordance with preoperative planning and user preferences. The at least one homerun wire 600 may be configured as a K-wire, guide wire, pin, and/or other similar device known in the orthopedic arts. The at least one homerun wire 600 may serve as a key component in achieving precise surgical outcomes by facilitating the alignment and resection of the distal femur. The modular PSI system 700, including the at least one homerun wire 600, may provide flexible trajectory options to simultaneously target multiple axes and depths for resection of a distal femur during a surgical procedure.

In a surgical procedure facilitated by the modular PSI system 700, the at least one homerun wire 600 may be strategically placed through a distal lateral PSI guide 710, and may allow customizable positioning and orientation of the at least one homerun wire 600. The modular PSI system 700 may utilize preoperative imaging data (e.g., CT or MRI scans) and user preferences to determine the ideal trajectory of the at least one homerun wire, which may include alignment with specific anatomical references or customized surgical criteria. The at least one homerun wire 600 may be placed thru a customized drill guide 720 which may be built onto the distal lateral PSI guide 710. Multiple parallel pins or wires may also be placed.

The trajectory of the at least one homerun wire 600 may vary based on surgeon preferences, AI-assisted recommendations, and/or necessary compromises to achieve optimal surgical outcomes. This flexibility may allow for a spectrum of preoperative planning scenarios based on specific targets, including, but not limited to: rotation based on surgical and/or anatomical TEA axis alignment, rotation based on perpendicularity to Whiteside's line, rotation based on PCA posterior condyle axis, rotation based on a Nguyen Messieh Line, rotation based on optimal patellar tracking, rotation based on a combination of two or more of the above rotational axes for optimal femoral component placement, posterior medial femur resection depth 603 of approximately 7.5 mm, distal femur valgus angle, matched with an implant size for posterior medial femoral condyle restoration, and/or distal femoral bone depth resection.

The versatility of the application of the at least one homerun wire 600 may be showcased through various preoperative scenarios. These scenarios may highlight the adaptability of the modular PSI system 700 to diverse surgical requirements and may ensure precision in the restoration of femoral anatomy and kinematics. The wire may also serve as a procedural anchor, allowing for real-time adjustments and ensuring fidelity to the surgical blueprint.

The following scenarios may illustrate the application in varying surgical contexts of the modular PSI system 700, each may underscore the flexibility and precision of the modular PSI system 700.

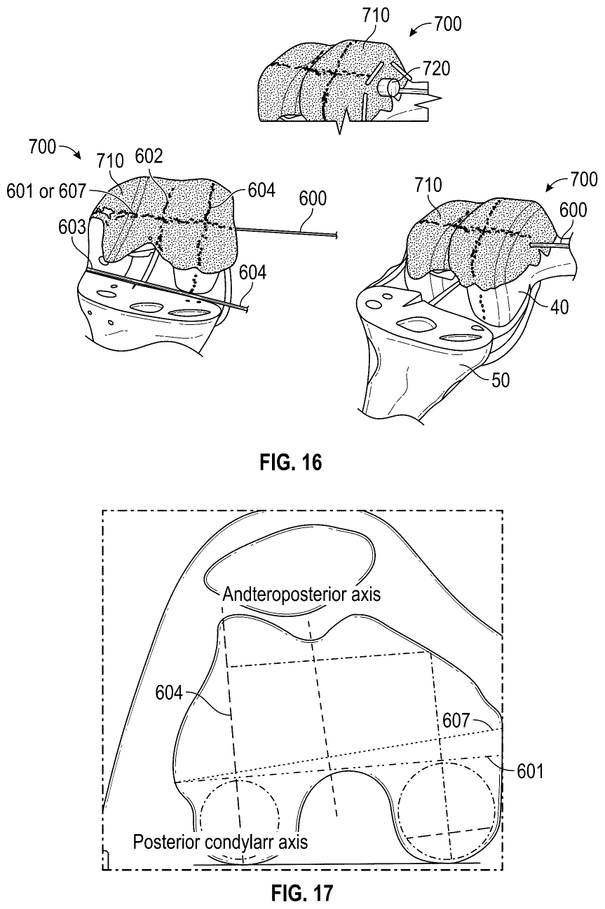

FIG. 16 shows three perspective views of an exemplary portion of a tibia and an exemplary portion of a femur indicating anatomical landmarks and a homerun wire according to an embodiment of the present disclosure.

Scenario 1: a homerun wire 600 may be aligned with the sTEA 601, perpendicular to Whiteside's line 602, approximately 10 mm from the distal femur, and/or distal femur valgus angulation as per user preference.

FIG. 17 is an annotated radiographic image of an exemplary portion of a tibia indicating anatomical landmarks according to an embodiment of the present disclosure.

Scenario 2: a homerun wire 600 may be aligned with a femoral cylindrical axis for kinematic and aligned with a distal femur resection depth based on resurfacing model criteria.

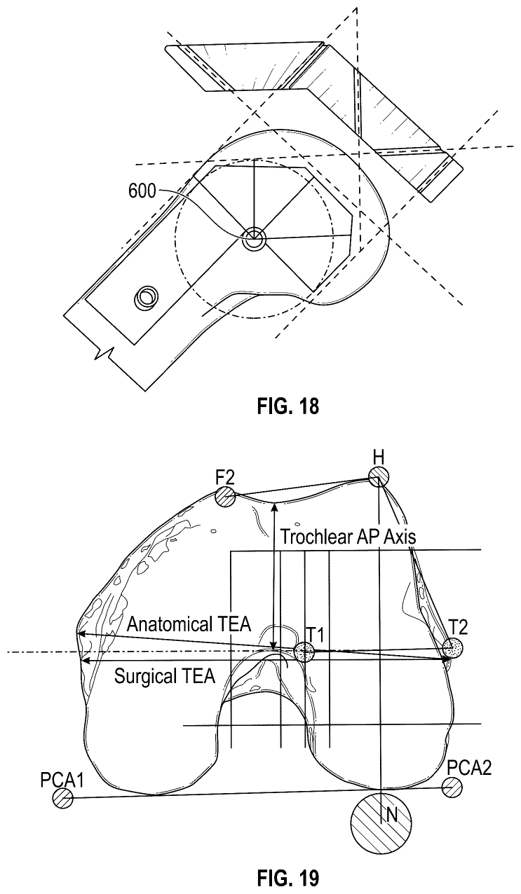

FIG. 18 is a graphical representation of a portion of a tibia indicating potential resection planes in relation to placement of a homerun wire according to an embodiment of the present disclosure.

Scenario 3: A homerun wire 600 may be aligned with a targeted distal femur angle, a distal femur resection level, and rotation based on user preference.

FIG. 19 is a graphical representation of a portion of a tibia indicating exemplary angles between various anatomical landmarks.

Scenario 4: a homerun wire 600 may be placed based on AI-suggested features.

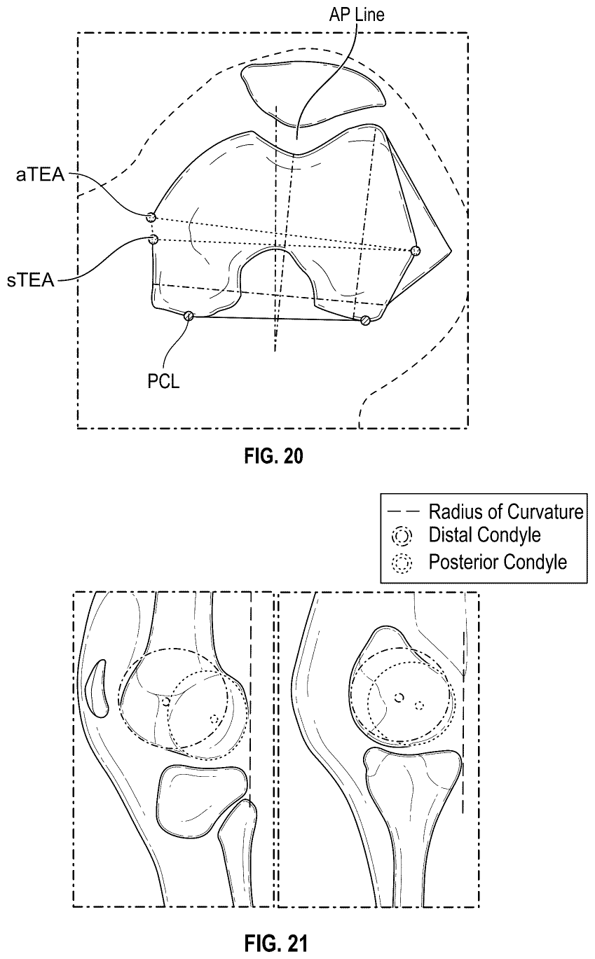

FIG. 20 is an annotated radiographic image of an exemplary portion of a tibia indicating anatomical landmarks according to an embodiment of the present disclosure. FIG. 20 shows an example for which a five-degree external rotation may achieve a best fit for patellar tracking, medial condyle offset and medical pivot. This is the anatomical TEA in the example shown. In the example the aTEA is also perpendicular to the Nguyen Messieh Line.

Scenario 5: A homerun wire 600 may be placed based on any other combination of angles and/or depths targeted. A surgeon may have the option to choose between various axes to set femoral rotation, including: Whiteside's line, anatomical TEA, surgical TEA, posterior condyle line PCA, and/or a Nguyen Messieh Line. The goal may be to set femoral rotation for patellar tracking, maintaining medial posterior condyle offset, creating a relative symmetrical gap balance with medial pivot and lateral translation, finding a best fit for femoral rotation, setting a distal valgus cut, and/or setting a distal femoral resection. A preoperative CT and/or MRI scan may preplan using an optimal set of axes to achieve these goals on a personalized approach.

FIG. 21 is an annotated radiographic image of an exemplary lateral femoral condyle and an exemplary medial femoral condyle.

The novel utility of the at least one homerun wire 600 may lie in its role as an anchor and procedural check to ensure that a surgical plan is being accurately executed, thereby reducing outliers. If adjustments are needed during the procedure, the modular PSI system 700 may be removed while the at least one homerun wire 600 remains in place. Additional wires may be inserted parallel to the at least one homerun wire 600 to accept cutting guides to complete a surgical procedure.

The at least one homerun wire 600 may be modified to be a Steinmann pin which may accept a manual or robotic saw to cut all surfaces of the femur from the lateral approach using the trajectory of the parallel wires for sliding the saw blade along the at least one homerun wire 600/Steinman pin and parallel wires.

The table below illustrates the various trajectory targets for the at least one homerun wire 600 placement to achieve targeted goals. The wire may serve to target multiple axes and depths of resections which may be impossible to perform with manual instruments.

| | Target | Description |
|---|---|---|
| 1 | Anatomical TEA (aTEA) | Alignment with anatomical transepicondylar axis |
| 2 | Surgical TEA (sTEA) | Alignment with surgical transepicondylar axis |
| 3 | Perpendicular to Whiteside's Line | Perpendicular alignment |
| 4 | Rotated to PCA (Posterior Condylar | Rotation with reference to PCA |
| | Axis) | |
| 5 | Perpendicular to Nguyen Messieh Line | Perpendicular alignment to Nguyen Messieh Line |
| 6 | Rotated in Reference to posterior | Restoration of femur offset |
| | condyle | |
| 7 | Valgus Distal Femur Resection | Valgus alignment for distal femur resection |

The modular PSI system 700, including the at least one homerun wire 600 and its flexible trajectory options to target multiple axes and depth of resection simultaneously, may represent a significant advancement in orthopedic surgery. Its precision, adaptability, and ability to reduce surgical outliers and reach desired targets make it an invaluable tool for enhancing patient outcomes in orthopedic procedures involving the distal femur.

A Nguyen Messieh Line 604 line may address a critical issue of femoral rotational malalignment in total knee arthroplasty (TKA), which has been a significant factor in the failure of these procedures. Numerous studies have highlighted the importance of proper rotational alignment of the femoral component in TKA, as it determines the position of the patellar groove and flexion gap stability. Despite the advancements in surgical techniques, the intraoperative determination of bony landmarks and identification of secondary reference axes remain challenging for surgeons.

Historical context, such as Berger's pioneering use of CT scans in 1993 to evaluate rotational alignment and define the surgical transepicondylar axis (sTEA) and posterior condylar angle (PCA), underscores the evolution of this field. Further studies have explored gender and ethnic-based differences in rotational alignment, the reliability of anatomical landmarks on CT scans, and the correlation between distal femur rotation and frontal alignment of the knee. These studies have consistently shown the variability in rotational axes and the significant impact of different patient populations. The collective research emphasizes the need for a method that accommodates these variations and offers a consistent and reliable reference for rotational alignment in TKA.

The Nguyen Messieh Line 604 may introduce a novel approach to determine optimal femoral rotation alignment in TKA. This novel axis, may be defined as the angle between the posterior condylar line and a line connecting the top of the lateral trochlea to the distal most posterior point of the middle of the lateral femoral condyle, may provide a reliable and consistent reference for rotational alignment. This method may not only complement existing rotational axes but may also offer significant improvements in terms of adaptability to a wide range of distal femoral morphologies and individual patient anatomies. The Nguyen Messieh Line 604 may be commonly known for its use in assessing total lateral condylar length rather than rotation. However, it may also be beneficial in its application for assessing rotation.

The Nguyen Messieh Line 604 may be versatile in its application, and may prove useful in preoperative planning, intraoperative guidance, and postoperative evaluation. It may be easily identified on CT and MRI scans, enhancing its utility across various surgical settings, including manual techniques, navigation and robotic-assisted surgeries. Additionally, this approach may allow for individualized rotational alignment based on gender, cultural background, and specific anatomical features, moving beyond generic assumptions and addressing the challenge of high variability in femoral anatomy. By providing a more accurate and personalized method for determining femoral rotation alignment, the Nguyen Messieh Line 604 may reduce the incidence of rotational malalignment and its associated complications, thereby improving patient outcomes in knee replacement surgeries.

The Nguyen Messieh Line 604 may represent a pivotal advancement in addressing rotational malalignment in total knee arthroplasty (TKA), a significant factor contributing to the failure of knee replacement surgeries. This innovative line may be easily identified on CT and MRI scans, enhancing its utility in both preoperative planning and postoperative evaluations.

The Nguyen Messieh Line 604 may be superimposed on a CT or MRI scan or placed directly onto the distal femur during surgery or determined by navigation. The positions of the aTEA and sTEA axes may be confirmed. The essential elements of the line may be the angle formed by the line from the apex of the lateral trochlea, to the inferior most middle point of the lateral femoral condyle and the PCA line. The medial and lateral femoral condyle may be outlined with spheres of larger and smaller diameters. A secondary Nguyen Messieh Line 604′ may be parallel to the first Nguyen Messieh Line.

Ease of Identification on Imaging: The Nguyen Messieh Line's unique definition, as the angle between the posterior condylar line and a line from the top of the lateral trochlea to the distal most posterior point of the middle of the lateral femoral condyle, may make it readily identifiable on CT and MRI scans. This feature may enable precise preoperative planning and accurate assessment of postoperative outcomes and may ensure optimal alignment of a femoral component.

Addressing Rotational Malalignment: Rotational malalignment remains a critical challenge in TKA, often leading to complications such as stiffness, instability, and early loosening of an implant. The Nguyen Messieh Line 604 may address this issue by providing a reliable and consistent reference for rotational alignment, crucial for positioning and sizing the components. Correct alignment is essential for maintaining loads on the interface and tension in the ligaments, thereby ensuring proper kinematic behavior and longevity of an implant.

Literature Review and Angular Relationships: A comprehensive review of literature on rotational alignment of the distal femur reveals the mean angular relationships between various rotation axes in the axial plane. The posterior condylar line, for instance, is on average 3° internally rotated relative to the surgical transepicondylar axis (sTEA), 5° relative to the anatomical transepicondylar axis (aTEA), and 4° relative to the perpendicular to the trochlear anteroposterior axis. The Nguyen Messieh Line 604 may take into account these angular relationships and the noted variability and standard deviations of reference axes.

Inter- and Intra-observer Variability: The Nguyen Messieh Line 604 may mitigate the challenges posed by inter- and intra-observer variability, particularly in locating the sTEA and/or aTEA during surgery. This variability has been a long-standing issue in achieving consistent rotational alignment in TKA.

Component Malrotation and Patellofemoral Malfunctioning: The degree of patellofemoral malfunctioning in TKA may be directly related to the amount of component internal rotation. Both excessive internal and external rotations of the femoral component may have detrimental effects. Excessive external rotation, for instance, may lead to symptomatic flexion instability, increased shear forces on the patella, and/or mechanical overload on the medial side of the joint. The Nguyen Messieh Line 604 may aid in avoiding these complications by providing a more precise rotational reference.

The Quest for an ‘Ideal’ Rotational Reference: Despite various attempts to define an ‘ideal’ rotational reference for TKA, the best rotational reference remains unclear. The Nguyen Messieh Line 604 may offer a novel approach in this regard and may provide a consistent and anatomically informed reference point for the rotation of the femoral component relative to landmarks on the distal femur.

The Nguyen Messieh Line 604 may be an innovative concept in total knee arthroplasty (TKA), revolutionizing the approach to determining the optimal rotational alignment of the femoral component. This line may be precisely defined as the angle between the posterior condylar line and a line connecting the top of the lateral trochlea to the distal most posterior point of the middle of the lateral femoral condyle. This distinct definition may set the Nguyen Messieh Line 604 apart, offering a consistent and reliable reference point that adapts to individual anatomical variations.

Crucially, the Nguyen Messieh Line 604 may account for the variability in the rotation axes of the distal femur among different individuals, addressing a significant challenge in TKA. This variability often complicates the intraoperative determination of bony landmarks, leading to inconsistencies in the alignment of the femoral component. By providing a more standardized and anatomically informed reference line, the Nguyen Messieh Line 604 may reduce the risk of rotational malalignment and its subsequent complications.

Furthermore, lines perpendicular to the Nguyen Messieh Line 604 may aid in identifying other key anatomical landmarks, such as the surgical transepicondylar line (sTEA). This feature may not only enhance the accuracy of the rotational alignment but may also contribute to a comprehensive understanding of the distal femur's morphology.

The adaptability of the Nguyen Messieh Line 604 may extend to its application in various surgical contexts. It may be used manually, digitally, or integrated within advanced surgical software and computer vision systems, demonstrating its versatility across different surgical techniques. Additionally, this line may prove invaluable in both primary TKA and revision surgery planning, offering a reliable method for assessing and planning corrective procedures.

In postoperative evaluations, the Nguyen Messieh Line 604 may be utilized in CT and MRI scans to assess femoral component alignment accurately. This application may be critical for confirming the success of the surgical procedure and for identifying any malalignment that may require correction.

By encompassing these wide-ranging applications, the Nguyen Messieh Line 604 may represent a significant leap forward in precision and patient-specific care in knee replacement surgeries. Its introduction may mark a pivotal moment in orthopedic surgery, aiming to enhance surgical outcomes, reduce complications, and cater to the unique anatomical needs of each patient.

The Nguyen Messieh Line 604 may represent a transformative advancement in total knee arthroplasty, providing a versatile and accurate method for determining optimal femoral rotation alignment. This innovative approach, defined precisely by anatomical landmarks, may offer a substantial improvement over existing techniques by facilitating more accurate implant positioning tailored to individual patient anatomy. Its adaptability to various distal femoral morphologies and compatibility with different surgical techniques and technologies, including manual, digital, and computer-assisted methods, may underscore its universal applicability.

Moreover, the utility of the Nguyen Messieh Line 604 may extend beyond initial surgical procedures, proving valuable in revision surgery planning and postoperative evaluations.

Its application in assessing femoral component alignment in postoperative CT and MRI scans may demonstrate its role as a critical tool in ensuring surgical success and patient satisfaction. By addressing the challenges of rotational malalignment, this invention may significantly enhance the precision of knee replacement surgeries, ultimately leading to improved patient outcomes and reduced risks of complications. The Nguyen Messieh Line 604 may stand as a significant contribution to the field of orthopedic surgery, embodying a commitment to innovation, patient-specific care, and surgical excellence.

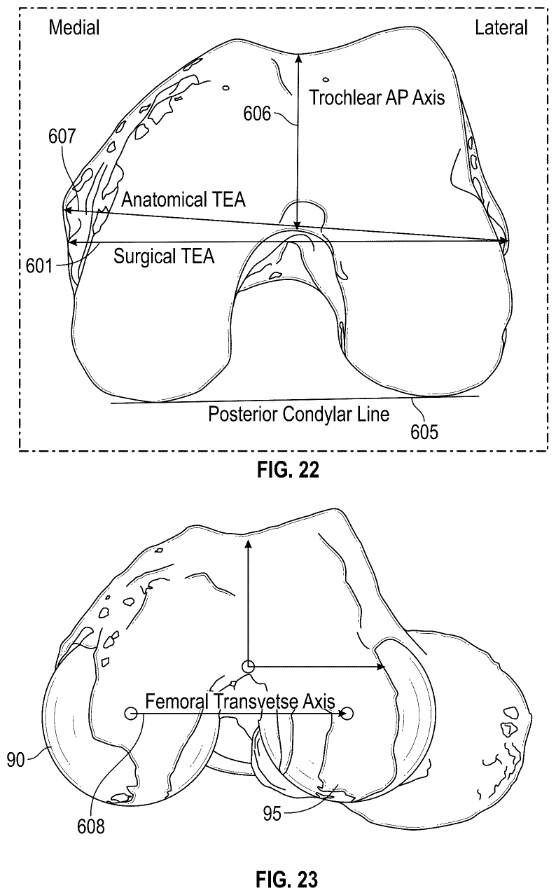

FIG. 22 is a superior view of an exemplary distal femur indicating anatomical landmarks. There are four axes currently used to assess femoral rotation: Trochlear AP axis 606, anatomical TEA (aTEA), surgical TEA (sTEA), and Posterior Condylar line.

FIG. 23 is a superior view of an exemplary distal femur indicating anatomical landmarks. A femoral transverse axis 608 as defined in the description of a Cartesian coordinate system of the femur. The femoral transverse axis 608 may connect the centers of the best-fit spheres to a medial condyle 90 and a lateral condyle 95. It may lie posterior to an aTEA 607 and may be parallel to a sTEA 601 in an axial plane. This may be considered a functional axis.

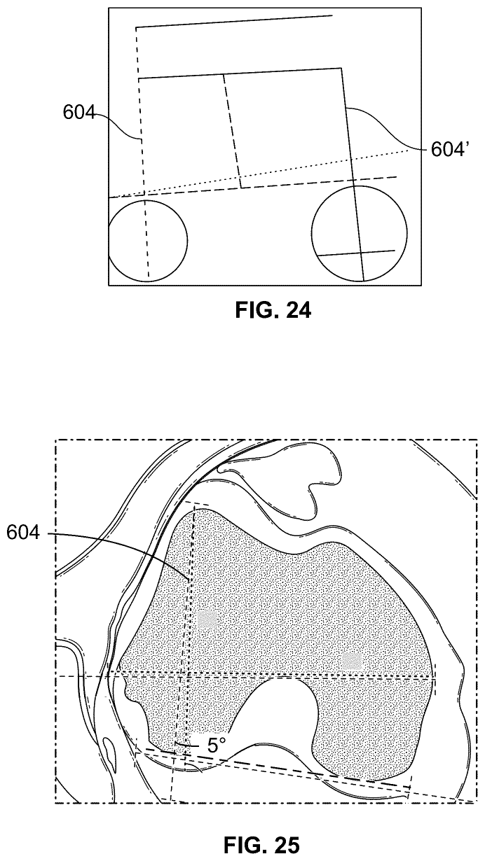

FIG. 24 is a section of a superior view of an exemplary distal femur indicating anatomical landmarks and a Nguyen Messieh Line 604 according to an embodiment of the present disclosure. The primary Nguyen Messieh Line 604 and a secondary Nguyen Messieh Line 604′ may be parallel to each other.

The Nguyen Messieh Line 604 may be easily used in addition with the other rotational axis to determine optimal femoral rotation alignment. The Nguyen Messieh Line 604 may be used manually, as a template, in conjunction with navigation and robotic surgery and revision planning. The Nguyen Messieh Line 604 may be used pre-operatively, intra-operatively and/or postoperatively.

The Nguyen Messieh Line 604 may be superimposed on a CT or MRI scan. The positions of the aTEA 607 and sTEA 601 axes may be confirmed through the CT scan. The essential elements of the Nguyen Messieh Line 604 may be the angle formed by the line from the apex of the lateral trochlea, to the inferior most middle point of the lateral condyle 95 and the posterior condylar line 605. The medial condyle 90 and lateral condyle 95 are outlined with spheres of larger and smaller diameter, respectively.

FIG. 25 is an annotated radiographic image of an exemplary distal femur indicating anatomical landmarks and a Nguyen Messieh Line 604 according to an embodiment of the present disclosure. The Nguyen Messieh Line 604 may help determine the aspect ratio of the distal femur; this is defined as the ML dimension of the distal femur divided by a length of the Nguyen Messieh Line 604. In the exemplary radiographic image of FIG. 25, the ML dimension of the distal femur is shown as 81 mm and the length of the Nguyen Messieh Line 604 is 68 mm. The aspect ratio of the exemplary femur, shown in the exemplary radiographic image, may be calculated as 81 mm/68 mm=1.2. The aspect ratio may vary with gender and different patient populations.

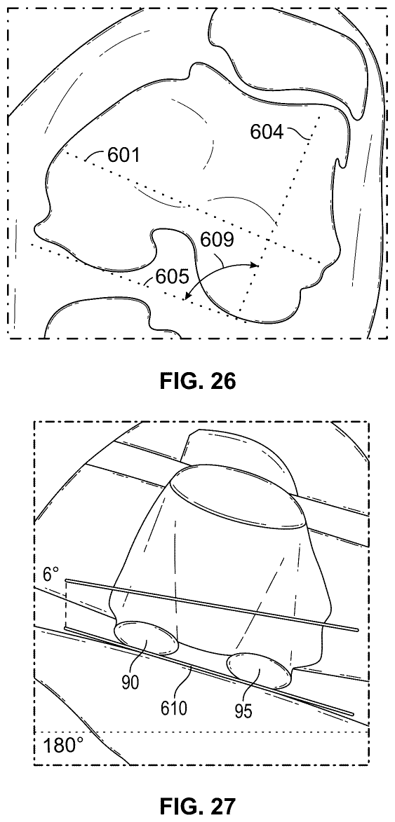

FIG. 26 is an annotated radiographic image of an exemplary distal femur indicating anatomical landmarks and a Nguyen Messieh Line according to an embodiment of the present disclosure. An angle 609 may be defined between the Nguyen Messieh Line 604 and the Posterior Condylar Line 605. The sTEA 601 may be perpendicular to the Nguyen Messieh Line 604. The sTEA 601 may be applied to a segmented femur for PSI preparation.

FIG. 27 is an annotated radiographic image of an exemplary distal femur indicating anatomical landmarks. Evaluation of axial alignment after TKA may require a CT scan with scatter reduction software to allow visualization of the medial condyle 90 and lateral condyle 95. The exemplary radiographic image of FIG. 27 shows a femoral component at 6° of internal rotation, relative to a surgical epicondylar axis 610.

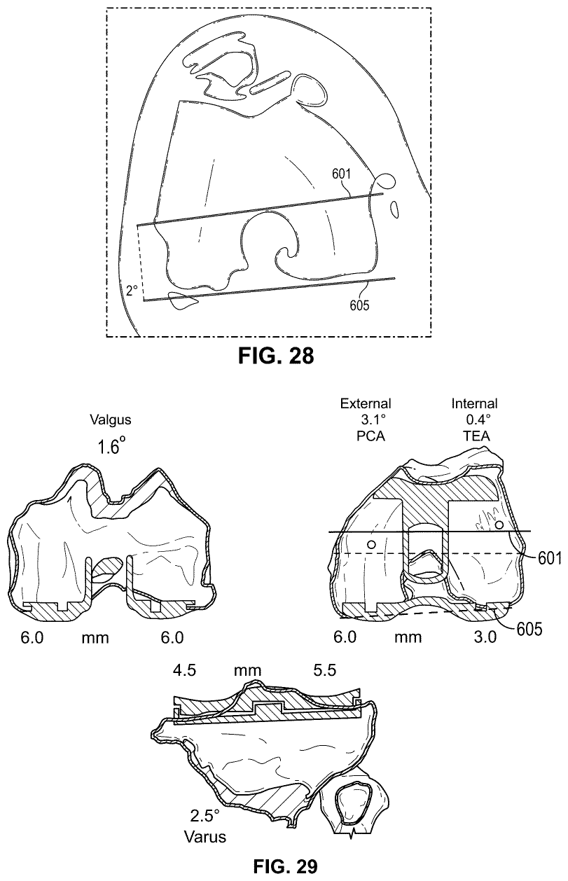

The Nguyen Messieh Line 604 may be used in a post-op CT scan to assess femoral component rotation. FIG. 28 is an annotated radiographic image of an exemplary distal femur indicating anatomical landmarks. FIG. 28 is an example of preoperative planning in a 62-year-old man with varus arthritis of his right knee. The angle between the sTEA 601 and the posterior condylar line 605, including osteophytes is calculated on the CT scan and implemented during surgery. In this exemplary case, the PCL was at 2° internal rotation, relative to the sTEA 601.

FIG. 29 is an example of a kinematically aligned robotic plan. The Nguyen Messieh Line 604 and secondary Nguyen Messieh Line 604′ may be used during robotic and navigated surgery to identify the sTEA 601. The Nguyen Messieh Line 604 may be applied to the distal femur planning and may confirm the location of the sTEA 601 and an external rotation with respect to the Posterior condylar line 605.

Based on previous research papers, the following mean angular relationships between the rotation axes of the distal femur in the axial plane may be calculated: the PCL may be on average 3° internally rotated relative to the sTEA, 5° relative to the aTEA and 4° relative to the trochlear AP Axis. The greatest interindividual variability is described for the trochlear AP axis. The worst track record regarding inter- and interobserver variability is for the TEA.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

The phrases “generally parallel” and “generally perpendicular” refer to structures that are within 30° parallelism or perpendicularity relative to each other, respectively. Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles of the disclosure.

The phrases “connected to,” “coupled to,” “engaged with,” and “in communication with” refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, as well as components that are removably and/or non-removably coupled with each other. The term “abutting” refers to items that may be in direct physical contact with each other, although the items may not necessarily be attached together. The phrase “fluid communication” refers to two or more features that are connected such that a fluid within one feature is able to pass into another feature. Moreover, as defined herein the term “substantially” means within +/−20% of a target value, measurement, or desired characteristic.

While specific embodiments and applications of the present disclosure have been illustrated and described, it is to be understood that the disclosure is not limited to the precise configuration and components disclosed herein. Various modifications, changes, and variations which will be apparent to those skilled in the art may be made in the arrangement, operation, and details of the methods and systems of the present disclosure without departing from its spirit and/or scope.

1. A ligament retractor system comprising:
a ligament retractor;
one or more sensors configured to identify a location of one or more anatomical landmarks or instruments; and
a processing unit configured to receive and process data from the one or more sensors.

2. The ligament retractor system of claim 1, further comprising a patient specific instrument (PSI) retractor attachment configured to be received by, and operatively engage, the ligament retractor, wherein the PSI retractor comprises the one or more sensors.

3. The ligament retractor system of claim 1, wherein the ligament retractor comprises a medial collateral ligament (MCL) retractor and the one or more anatomical landmarks comprises at least one of a tibial plateau and a soft tissue adjacent to the tibial plateau.

4. The ligament retractor system of claim 1, wherein the one or more sensors comprises an inertial measurement unit (IMU) sensor configured to obtain positional data of the ligament retractor.

5. The ligament retractor system of claim 1, wherein the one or more sensors comprises an optical sensor configured to provide 3D visualization data of an anatomical landmark proximate the ligament retractor.

6. The ligament retractor system of claim 1, wherein the one or more sensors comprises a Hall effect sensor configured to detect proximity of a metallic surgical instrument to the ligament retractor.

7. The ligament retractor system of claim 1, wherein the ligament retractor comprises the one or more sensors.

8. A ligament retractor system comprising:
a ligament retractor comprising one or more sensors; and
a processing unit configured to receive and process data from the one or more sensors;
wherein the one or more sensors are configured to detect proximity of a cutting device to the one or more sensors.

9. The ligament retractor system of claim 8, wherein the one or more sensors comprise one or more Hall effect sensors.

10. The ligament retractor system of claim 8, wherein the cutting device comprises a material comprising a magnetic field detectable by the one or more sensors.

11. The ligament retractor system of claim 8, further comprising a cutting guide, wherein the cutting guide is configured to be coupled to the ligament retractor and to receive and guide the cutting device.

12. The ligament retractor system of claim 8, further comprising a patient specific instrument (PSI) retractor attachment configured to be received by, and operatively engage, the ligament retractor.

13. The ligament retractor system of claim 12, further comprising a cutting guide, wherein the cutting guide is configured to be coupled to the PSI retractor attachment and to receive and guide the cutting device.

14. The ligament retractor system of claim 8, wherein the ligament retractor comprises a medial collateral ligament (MCL) retractor.

15. The ligament retractor system of claim 8, further comprising a bone saw comprising a saw blade and a battery pack configured to provide electric voltage to power the bone saw, wherein:
the battery pack comprises an indicator and a circuit board configured to receive a trigger voltage from the one or more sensors;
the one or more sensors are configured to communicate the trigger voltage to the circuit board upon detection of the proximity of the saw blade;
the circuit board is configured to activate the indicator upon receipt of the trigger voltage; and
the circuit board is further configured to switch off the electric voltage from the battery pack to the bone saw upon receipt of the trigger voltage.

16. A ligament retractor system comprising:
a ligament retractor comprising one or more sensors; and
a processing unit configured to receive and process data from the one or more sensors;
wherein the one or more sensors are configured to confirm placement of the ligament retractor during a surgical procedure.

17. The ligament retractor system of claim 16, wherein the one or more sensors comprise one or more inertial measurement unit (IMU) sensors configured to obtain positional data of the ligament retractor.

18. The ligament retractor system of claim 16, wherein the ligament retractor comprises a medial collateral ligament (MCL) retractor.

19. The ligament retractor system of claim 16, wherein the ligament retractor further comprises a Hall effect sensor and the ligament retractor system further comprises a cutting device, wherein the Hall effect sensor is configured to detect a proximity of the cutting device to the ligament retractor.

20. The ligament retractor system of claim 19, wherein the cutting device comprises a material comprising a magnetic field detectable by the one or more sensors.

21. A ligament retractor system comprising:
a ligament retractor configured to retract a ligament during a surgical procedure; and
a patient specific instrument (PSI) retractor attachment configured to be received by, and operatively engage, the ligament retractor;
wherein the PSI retractor attachment is configured to engage an anatomical landmark.

22. The ligament retractor system of claim 21, wherein the ligament retractor comprises one or more sensors configured to identify a location of one or more anatomical landmarks or instruments.

23. The ligament retractor system of claim 21, wherein the PSI retractor attachment comprises one or more sensors configured to identify a location of one or more anatomical landmarks or instruments.

24. The ligament retractor system of claim 21, further comprising a cutting guide, wherein the cutting guide is configured to be coupled to the PSI retractor attachment and to receive and guide a cutting device.
